(19) United States
(12) Patent Application Publication
(10) Pub. No.: US 2024/0090854 A1
(43) Pub. Date: Mar. 21, 2024

(54) WEARABLE MEDICAL DEVICE SYSTEM WITH CAREGIVER NOTIFICATIONS

(71) Applicant: West Affum Holdings Corp., Grand Cayman, KY (US)

(72) Inventors: Sarah A. Bishop, Chesterfield, MO (US); Anne E. Mothersbaugh, Royal Oak, MI (US); Kimberley L. Malone, Monroe, MI (US)

(21) Appl. No.: 17/946,913

(22) Filed: Sep. 16, 2022

(51) Int. Cl. A61B 5/00; A61B 5/11

(52) U.S. Cl. CPC A61B 5/7465; A61B 5/0022; A61B 5/1112; A61B 5/742

(57) ABSTRACT

A wearable medical device caregiver notification system includes a patient communication device. The patient communication device is configured to receive patient data from the wearable medical device. Using a criteria app, the patient communication device can determine if preset goal criteria, such as wear time compliance or number of steps taken, have been met. Upon determining that a goal criterion has not been met, the patient communication device can transmit a notification to a designated caregiver communication device indicating that the goal was not achieved.

### BACKGROUND

Wearable medical devices, such as wearable cardioverter defibrillators (WCDs) collect and process cardiac signal into an electrocardiogram (ECG) data. WCDs are configured to analyze patient signals to determine if the patient is experiencing a cardiac event. For example, if a ventricular tachycardia/ventricular fibrillation (VT/VF) arrest occurs, the WCD detects the event and alarms the patient and bystanders of an impending shock. If the patient does not respond to the alarms, the WCD delivers a therapeutic shocks to the patient.

In some instances, a WCD can also process other patient data. For example, activity signals can be processed into step count data and/or wear time data. The patient data can then be used to provide notifications to the patient's designated caregiver.

### BRIEF DESCRIPTION OF THE DRAWINGS

FIG. 1 is a diagram of a notification system including a wearable cardiac defibrillator (WCD), a patient communication device, a remote monitoring system, and a caregiver communication device, in accordance with one or more embodiments.

FIG. 2 is a diagram of a patient communication device of FIG. 1A in accordance with embodiments.

FIG. 3 is a diagram of a caregiver communication device in accordance with one or more embodiments.

FIG. 4 is a flow diagram of a method of transmitting a notification to a caregiver communication device in accordance with one or more embodiments.

FIG. 5 is a flow diagram of a method of transmitting a notification to a designated caregiver communication device in accordance with one or more embodiments.

FIG. 6 is a flow diagram of for transmitting a notification to a designated caregiver communication device in accordance with one or more embodiments.

FIG. 7 is a flow diagram of a method of transmitting a notification to a designated caregiver communication device in accordance with one or more embodiments.

It will be appreciated that for simplicity and/or clarity of illustration, elements illustrated in the figures have not necessarily been drawn to scale. For example, the dimensions of some of the elements can be exaggerated relative to other elements for clarity. Further, if considered appropriate, reference numerals have been repeated among the figures to indicate corresponding and/or analogous elements.

### DETAILED DESCRIPTION

In the following detailed description, numerous specific details are set forth to provide a thorough understanding of claimed subject matter. It will, however, be understood by those skilled in the art that claimed subject matter can be practiced without these specific details. In other instances, well-known methods, procedures, components and/or circuits have not been described in detail.

In the following description and/or claims, the term “and/or” can mean “and”, it can mean “or”, it can mean “exclusive-or”, it can mean “one”, it can mean “some, but not all”, it can mean “neither”, and/or it can mean “both”, although the scope of claimed subject matter is not limited in this respect. In the following description and/or claims, the terms “comprise” and “include,” along with their derivatives, can be used and are intended as synonyms for each other.

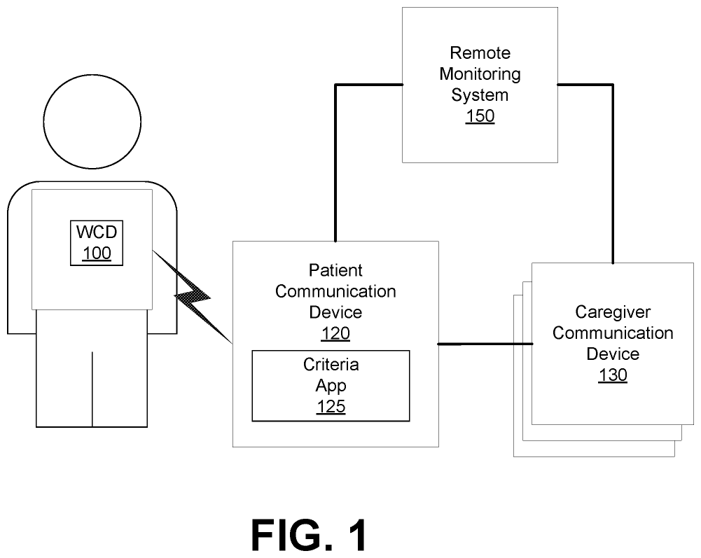

Referring now to FIG. 1, a diagram of a notification system including a wearable cardioverter defibrillator in accordance with one or more embodiments will be discussed. FIG. 1 illustrates a system comprising a wearable cardioverter defibrillator (WCD) 100, a remote monitoring system 150, a caregiver communication device 130, and a patient communication device 120, wherein the patient communication device 120 includes a criteria app 125.

Description of a WCD, similar to the WCD 100, can be found in U.S. Patent App. No. US2019/0209853 and/or U.S. Pat. No. 11,235,143, both of which is incorporated herein by reference in their entirety. The WCD system includes a number of components. These components can be provided separately as modules that can be interconnected, or can be combined. The WCD can interact with devices, such as a patient communication device 120, which in turn can interact with other systems and devices, such as a remote monitoring system 150, and or a caregiver communication device 130.

Other embodiments of a WCD include an adhesive structure or another way for attaching to the patient. In some embodiments, support structure can include a container or housing, which can even be waterproof. In such embodiments, the support structure can be worn by being attached to the patient's body by adhesive material, for example as shown and described in U.S. Pat. No. 8,024,037. Support structure can even be implemented as described for the support structure of US Pat. App. No. US2017/0056682, which is incorporated herein by reference. Of course, in such embodiments, the person skilled in the art will recognize that additional components of the WCD system can be in the housing of a support structure instead of being attached externally to the support structure, for example as described in the US2017/0056682 document. There can be other examples.

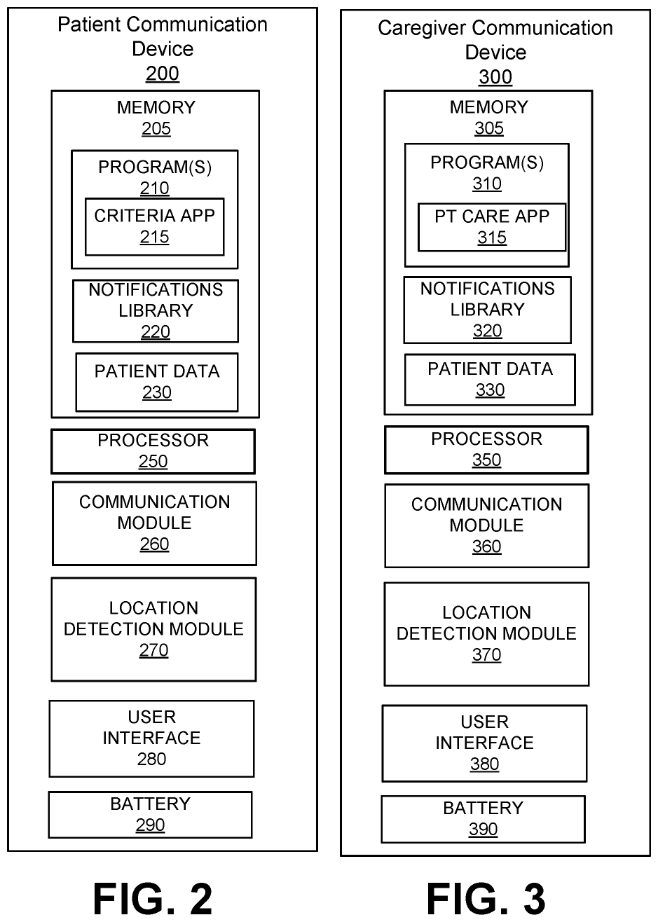

The WCD as shown and described in above-referenced publications includes a processor. Similarly, the WCD 100 includes a processor, which is configured to process patient output signals into patient data including an electrocardiogram (ECG) data and/or heart rate data, wear time data, and/or step count data. The WCD's communication module can wirelessly, for example by way of Bluetooth, transmit the processed patient data out of the WCD to another device, such as the patient communication device 120. In other embodiments, the WCD's communication module can communicate with the remote monitoring system 150 and/or caregiver communication device via Wi-Fi, cellular and/or other communication networks. One embodiment of the patient communication device 120, is the patient communication device as shown in FIG. 2. The remote monitoring system 150 can be a centralized monitoring station managed by clinical and technical experts, a medical response system, and so on.

In some embodiments, the patient communication device 120, upon receiving the WCD-processed patient data, using the criteria app 125, can determine if goal criteria, such as wear time compliance or number of steps taken, have been met. Upon determining that a goal criterion or goal criteria has/have not been met, the patient communication device 120 can send a notification to a caregiver communication device 130 notifying the goal was not achieved.

In some embodiments, notification criteria comprise detection of an arrhythmia in the ECG data, or for example, ECG data a cardiac signal indicative that the patient wore the WCD less than 80% of the wear time goal. In some embodiments, notification criteria comprise the patient not taking a minimum preset number of steps per day. In some embodiments, notification criteria comprise information about the patient not wearing the WCD at least 18 hours per day, for example.

In some embodiments, the remote monitoring system 150 can be configured to receive a notification from the patient communication device 120, determine the location of the patient communication device 120, and display the patient communication device's 120 location. In some examples, the remote monitoring system can comprise the Kestra CareStation (™) platform from Kestra Medical Technologies of Kirkland, Washington, USA.

The patient communication device 120 can be a wireless telephone, a smartphone, a Personal Digital Assistant (PDA), a personal electronic device, a pager, a laptop computer, a tablet, an e-reader, and so on. It can have an application, or “app”, made according to embodiments, so as to perform various functions as described. In such embodiments, patient communication device 120 can communicate with a wireless service provider network. Furthermore, the location of the WCD 100 can be determined using Global Positioning System (GPS) for example using appropriate hardware in the patient communication device 120. Upon pairing of the WCD 100 and the patient communication device 120, GPS information can become known and communicated to the remote monitoring system 150, and/or the caregiver communication device 130, and/or Emergency Medical Services (EMS) services. The patient communication device 120 can provide a communication path for the patient data from the WCD 100. The patient communication device 120 can also be used to transmit notifications to a designated caregiver device 130 based on the patient data from the WCD 100.

The caregiver communication device 130 can be used to transmit notifications from the patient communication device 120 and/or from the remote monitoring system 150 to inform a caregiver of the patient's status, request a troubleshooting assistance, provide motivational feedback, and the like.

In some embodiments, the caregiver communication device 130 can provide location information to the remote monitoring system 150 to enable the remote monitoring system 150 to track the location of the caregiver communication device 130. In some embodiments, the remote monitoring system 150 can be configured to query the caregiver communication device to provide the caregiver communications device's location. For example, a notification from the patient communication device 120 to the remote monitoring system 150 can trigger the remote monitoring system 150 to query caregiver communication device 130 for its location.

In some embodiments, the caregiver communication device 130 is configured to provide instructions and/or suggestions to the caregiver for checking on the patient. Suggestions or instructions may include steps on what to do in some situations and/or how to provide care. Some embodiments may include suggestions for situations where the patient needs encouragement, emotional and/or physical attention. Some embodiments may include instructions situations that require emergency attention, for example, CPR administration, etc. Instructions and/or suggestions may also include troubleshooting a lack of WCD wear or step goal compliance.

In some embodiments, the caregiver communication device 130 may include a notifications library 320 or database with a default instruction or suggestion options. In some embodiments, instructions on the caregiver communication device 130 comprise an instruction for the designated caregiver to send a message to the remote monitoring system 150.

In some embodiments, instead of the caregiver communication device being configured with instructions and/or suggestions, the remote monitoring system 150 is configured to send one or more messages to the designated caregiver communication device 130. The one or more messages can comprise, for example, one or more instructions to check on the patient, suggestions or instructions on what to do in particular situations, and/or instructions on how to provide care. In some embodiments, the remote monitoring system 150 can be configured to determine from the received notifications a type of situation such as, for example, situations where the patient needs encouragement, emotional and/or physical attention, and/or situations where in the patient needs emergency attention such as vital signs check and/or CPR administration, etc. Instructions and/or suggestions may also include troubleshooting a lack of WCD wear or step goal compliance.

In some embodiments, instructions include an augmented reality instructions and/or provide audio, visual, and/or touch guidance on steps to take to, for example, administer a CPR or put on the WCD, etc.

The caregiver communication device 130 can be a wireless telephone, a smartphone, a Personal Digital Assistant (PDA), a personal electronic device, a pager, a laptop computer, a tablet, an e-reader, and so on. It can have an application, or “app”, made according to embodiments, so as to perform various functions as described. In such embodiments, caregiver communication device 130 can communicate with a wireless service provider network. Furthermore, the location of the patient communication device 120 can be determined using Global Positioning System (GPS) for example using appropriate hardware in the patient communication device 120 and in the caregiver communication device 130. In some embodiments, upon receiving a notification from the patient communication device 120, GPS information can become known and communicated to the caregiver communication device 130, and/or the remote monitoring system 130, and/or Emergency Medical Services (EMS) services.

FIG. 2 is a block diagram of a patient communication device 200 according to embodiments of the present disclosure. Embodiments of patient communication device 200, which can be used to implement patient communication device 120, (FIG. 1) includes: a battery 290; a user interface 280 configured for patient/user interaction; a location detection module 270 configured to determine the location of the device 200; a communication module 260 configured to transmit data; a processor 250, and a memory 205 storing program(s) 210.

In embodiments, processor 250 is configured to process received data using program(s) 210 and provide notifications selected from notifications library 220 stored in memory 205. Program(s) 210 includes one or more programs, such as for example a criteria app 215. Processor 250 can also be configured to store in memory 205 Patient Data 230 (“Data”), such as ECG data and/or step count data (not shown) received from the WCD 100 (FIG. 1).

In some embodiments, the patient communication device 200 is configured to receive Patient Data 230 such as ECG data and/or the step count data from the WCD 100 via the communication module 260 and store the received data in memory 205. The communication module 260 of the patient communication device 200 is further configured to transmit the Patient Data 230 to the remote monitoring system 150, shown in FIG. 1. The criteria app 215 is configured to: determine whether the Patient Data 230, such as the ECG data or the step count data, or both, do not meet one or more predetermined notification criteria; and when so confirmed, to transmit a notification selected from notifications library 220 to the caregiver communication device 130 (FIG. 1).

For example, the criteria app 215 can be configured to determine whether the received Patient Data 230 do not meet a predetermined goal, such as wear time of more than n hours per day; and/or the step count data do not meet a predetermined goal, such as more than x number of steps per day. Responsive to determining that a goal has not been met, processor 250 is configured to cause the communication module 260 of the patient communication device 200 to transmit a notification from notification library 220 to caregiver communication device such as caregiver communication device 130 (FIG. 1) or caregiver communication device 300 (FIG. 3).

In some embodiments, the communication module 260 can also send the Patient Data 230 to a remote monitoring system, such as the remote monitoring system 150 shown in FIG. 1. The Patient Data 230 can include the ECG data and/or step count data and/or at least an indication of the notification sent to the caregiver communication device 300.

FIG. 3 is a block diagram of a caregiver communication device 300, according to embodiments. Embodiments of caregiver communication device 300, which can be used to implement caregiver communication device 130 (FIG. 1), includes: a battery 390; a user interface 380 configured for caregiver/user interaction, a location detection module 370 configured to determine the location of the device 300, a communication module 360 configured to transmit data; a processor 350; and a memory 305 storing program(s) 310.

In embodiments, processor 350 is configured to process received data using program(s) 310 and to provide notifications selected from notifications library 320. Program(s) 310 include one or more programs, such as for example a patient caregiver app 315. Processor 350 can also be configured to store in memory 305 the Patient Data 330 such as ECG and/or step count data (not shown) received from the patient communication device 200 (FIG. 2).

In one embodiment, patient care app 315 can be made available to a designated caregiver. The patient care app 315 can be a downloadable app, which can be downloaded to a designated caregiver's mobile device, for example. A designated caregiver can be a lay person, a family member, a friend, a neighbor, or anyone the patient wants to designate. A designated caregiver is a person willing to support and/or care for patient who is not meeting certain goal criteria, such as wear time, or steps, or both. In some embodiments, more than one designated caregiver can be assigned to a patient. In some embodiments, the patient care app 315 can be utilized by another current or former WCD wearer, who may be going or have gone through the experience and can be designated to encourage and support a fellow patient. In one embodiment, the caregiver communication device 300 can receive a notification from a patient communication device such as the patient communication device 200, shown in FIG. 2. In response to receiving the notification, the patient care app 315 is configured to inform a designated caregiver.

In some embodiments, additional information notification can also be transmitted to the caregiver communication device 300 by the patient communication device and/or a remote monitoring system such as remote monitoring system 150 (FIG. 1). The additional information can include sleep criteria, alerts patient received, shock events, patient triggered episodes, arrhythmia detection, etc.

The patient care app 315 can promote a sense of connection between patients, family members, friends, fellow WCD wearers, etc. This sense of connection can be used by these parties to help patients meet wear time and wellness goals. These goals can include a patient taking a preset number of steps per day, meeting sleep quality, and/or other goals.

In some embodiments, the caregiver communication device 300 sends a request to the patient communication device for the patient communication device's location. The caregiver communication device can be configured to send such a location request when, for example, the patient has not been responsive to communications sent to the patient communication device. In addition, some embodiments of the caregiver communication device may be configured to send a location request to the patient communication device responsive to receiving a notification that may indicate the patient needs in-person assistance. The caregiver will then be able to go (or send emergency response personnel) to the location of the patient communication device. In some situations, it can also be helpful for medical personnel to locate the patient communication device to contact a designated family member, a neighbor, or a friend to go to the patient for wellness check, and/or to deliver information if an emergency occurs.

The devices and/or systems mentioned in this document may perform functions, processes, acts, operations, actions and/or methods. These functions, processes, acts, operations, actions and/or methods may be implemented by one or more devices that include logic circuitry. A single such device can be alternately called a computer, and so on. It may be a standalone device or computer, such as a general-purpose computer, or part of a device that has and/or can perform one or more additional functions. The logic circuitry may include a processor and non-transitory computer-readable storage media, such as memories, of the type described elsewhere in this document. Often, for the sake of convenience only, it is preferred to implement and describe a program as various interconnected distinct software modules or features. These, along with data are individually and also collectively known as software. In some instances, software is combined with hardware, in a mix called firmware.

Moreover, methods and algorithms are described below. These methods and algorithms are not necessarily inherently associated with any particular logic device or other apparatus. Rather, they are advantageously implemented by programs for use by a computing machine, such as a general-purpose computer, a special purpose computer, a microprocessor, a processor such as described elsewhere in this document, and so on.

This detailed description may include flowcharts, display images, algorithms, and symbolic representations of program operations within at least one computer readable medium. An economy may be achieved in that a single set of flowcharts can be used to describe both programs, and also methods. So, while flowcharts describe methods in terms of boxes, they may also concurrently describe programs.

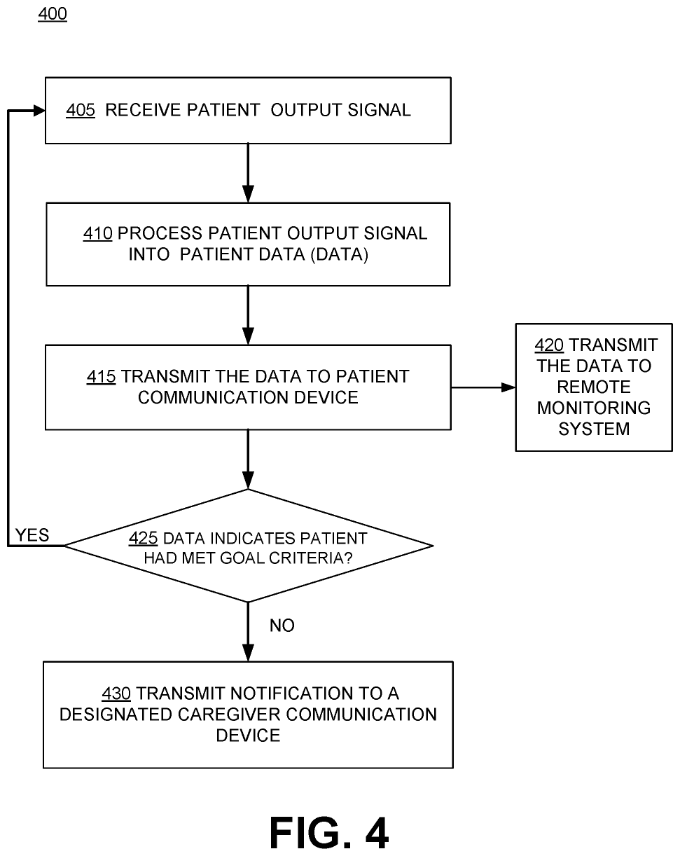

Referring now to FIG. 4, a flowchart is shown illustrating an embodiment of a process 400 for transmitting a notification to a designated caregiver communication device. For example, the caregiver communication device can be similar to the caregiver communication device 300 (FIG. 3) or caregiver communication device 130 (FIG. 1).

In a step 405, the process receives a patient output signal. In some embodiments, the patient output signal comprises a patient physiological signal (e.g., ECG signal) and/or activity signal (e.g., motion, steps) and/or environmental signal (e.g., location, ambient temperature, altitude, etc.) sensed by one or more sensors of a wearable medical device such as a WCD. For example, the WCD may be similar to WCD 100 (FIG. 1).

In a step 410, the patient output signal is processed into Patient Data. In some embodiments, the patient output signal is processed into Patient Data by a processor of the wearable medical device. For example, the patient output signal may be processed into electrocardiogram (ECG) data and/or heart rate data, and/or other physiological data. In some embodiments, the patient output signal may be processed into activity data such as, for example, step count data. The Patient Data is also referred to in process 400 as Data.

In a step 415, the Data is transmitted to a patient communication device. In some embodiments, the Data is transmitted by a communication module of the wearable medical device. The patient communication device can be similar to, for example, patient communication device 120 (FIG. 1) or patient communication device 200 (FIG. 2). In some embodiments, a wearable medical device such as a WCD transmits the Data using a Bluetooth connection with the patient communication device.

In a step 420, the Data is transmitted to a remote monitoring system by the patient communication device. In some embodiments, the remote monitoring system is similar to, for example, the remote monitoring system 150 (FIG. 1).

In a step 425, the Data is analyzed to determine if the Data indicates the patient met one or more goal criteria. In some embodiments, a processor of patient communication device makes this determination. For example, the patient communication device can have a processor running a criteria app similar to, for example, processor 250 and criteria app 215 of patient communication device 200 (FIG. 2). If the Data is determined to indicate the patient had met criteria goal, process 400 returns to step 405 to receive another patient output signal. However, if the Data is determined to indicate that the patient has not met the criteria goal, process 400 proceeds to a step 430.

In a step 430, a notification is transmitted to a designated caregiver communication device. In some embodiments, the patient communication device in configured with contact information of one or more caregiver communication devices and can determine which caregiver communication device is designated to receive the notification. The designated caregiver communication device can be similar to the caregiver communication device 130 (FIG. 1) or the caregiver communication device 300 (FIG. 3).

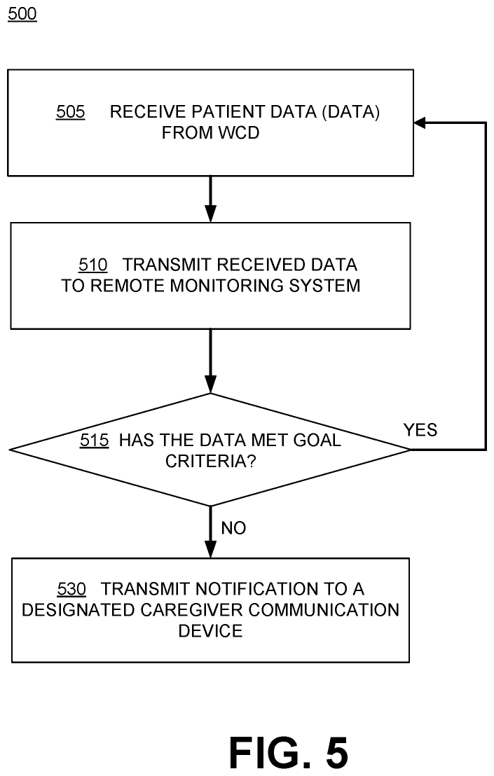

FIG. 5 is a flowchart illustrating a process 500 for transmitting a notification to a designated caregiver communication device, according to embodiments. The caregiver communication device can be similar to, for example, the caregiver communication device 300 (FIG. 3) or caregiver communication device 130 (FIG. 1).

In a step 505, Patient Data is received. In some embodiments, the Patient Data is received by a patient communication device. The patient communication device can be similar to, for example, the patient communication device 200 (FIG. 2) or the patient communication device 120 (FIG. 1). In some embodiments, the Patient Data is received from a wearable medical device. For example, the wearable medical device can be a WCD similar to WCD 100 (FIG. 1). The Patient Data is also referred to in FIG. 5 as “Data”

In a step 510, the Data is transmitted to a remote monitoring system. In some embodiments, the Data is transmitted by a communication module similar to communication module 260 (FIG. 2). In some embodiments, the remote monitoring system can be similar to, for example, the remote monitoring system 150 (FIG. 1).

In step 515, the Data is also analyzed to determine if the Data indicates the patient met one or more goal criteria. In some embodiments, a processor of the patient communication device makes such a determination. For example, the patient communication device can have a processor running a criteria app similar to processor 250 and criteria app 215 of the patient communication device 200 (FIG. 2). Upon determining that the Data indicates the patient had met the one or more goal criteria, process 500 returns to step 505 to receive more Patient Data. However, upon determining the Data indicates that the patient has not met the one or more goal criteria, process 500 proceeds to a step 530.

In step 530, a notification is transmitted to the designated caregiver communication device. In some embodiments, the patient communication device in configured with contact information of one or more caregiver communication devices and can determine which caregiver communication device is designated to receive the notification. The designated caregiver communication device can be similar to the caregiver communication device 130 (FIG. 1) or the caregiver communication device 300 (FIG. 3).

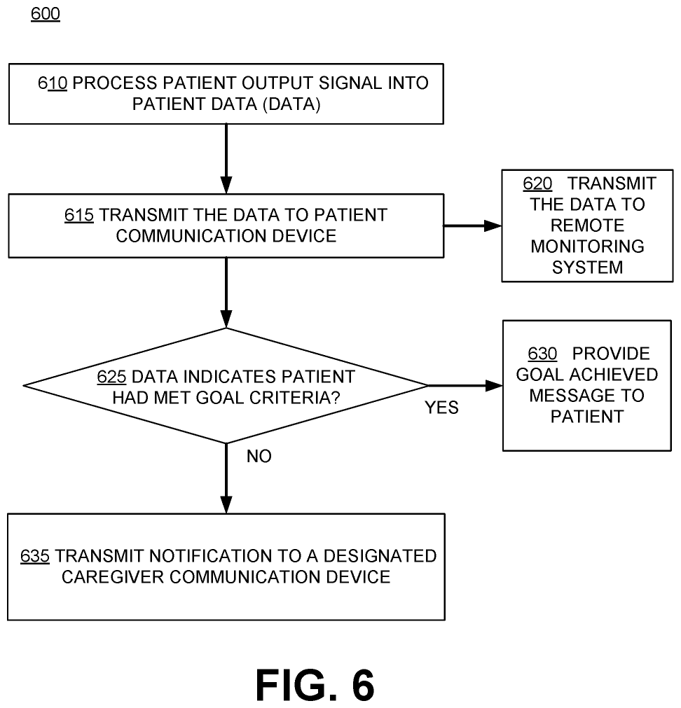

FIG. 6 is a flowchart illustrating a process a process 600 for transmitting a notification to a designated caregiver communication device, according to embodiments. The designated caregiver communication device can be similar to, for example, the caregiver communication device 300 (FIG. 3) or caregiver communication device 130 (FIG. 1). In some embodiments, the notification can be transmitted by a patient communication device. The patient communication device can be similar to, for example, the patient communication device 200 (FIG. 2) or the patient communication device 120 (FIG. 1)

In a step 610, a patient output signal is processed into Patient Data, also referred to as “Data.” In some embodiments, the patient output signal comprises a patient physiological signal (e.g., ECG signal) and/or activity signal (e.g., motion, steps) and/or environmental signal (e.g., location, ambient temperature, altitude, etc.) sensed by one or more sensors of a wearable medical device such as a WCD. For example, the WCD may be similar to WCD 100 (FIG. 1). In some embodiments, the patient data is processed by a processor of the wearable medical device.

In step 615, the Data is transmitted to a patient communication device. In some embodiments, the Data is transmitted by a communication module (not shown) of the wearable medical device.

In step 620, the Data may also be transmitted to a remote monitoring system. In some embodiments, the Data is transmitted to the remote monitoring system by a communication module of the patient communication device similar to, for example, communication module 260 (FIG. 2). In some embodiments, the remote monitoring system is similar to, for example, the remote monitoring system 150 (FIG. 1).

In step 625, the Data is analyzed to determine that the patient has met one or more goal criteria. In some embodiments, a processor of the patient communication device makes this determination. For example, the patient communication device can have a processor running a criteria app similar to, for example, processor 250 and criteria app 215 of patient communication device 200 (FIG. 2).

Upon determination that the patient has met the one or more goal criteria, the process 600 proceeds to a step 630 in which a message is sent to the patient that a goal has been achieved. In some embodiments, the message is sent using a communication module of the patient communication device similar to, for example, communication module 260 (FIG. 2). In some embodiments, the patient communication device can be configured to display a message to the patient about meeting the goal, for example—“Goal Met! You wore the WCD for 20 hours in the last 24 hours! You took 10000 steps! Good Job,” or something to that effect.

However, upon determining in step 625 that the one or more goal criteria has not been met, the process 600 proceeds to a step 635. In step 635, a notification is sent to the designated caregiver device to inform a caregiver that the patient is not meeting the one or more goal criteria. In some embodiments, the notification is sent by a communication module of the patient communication device similar to, for example, communication module 260 (FIG. 2).

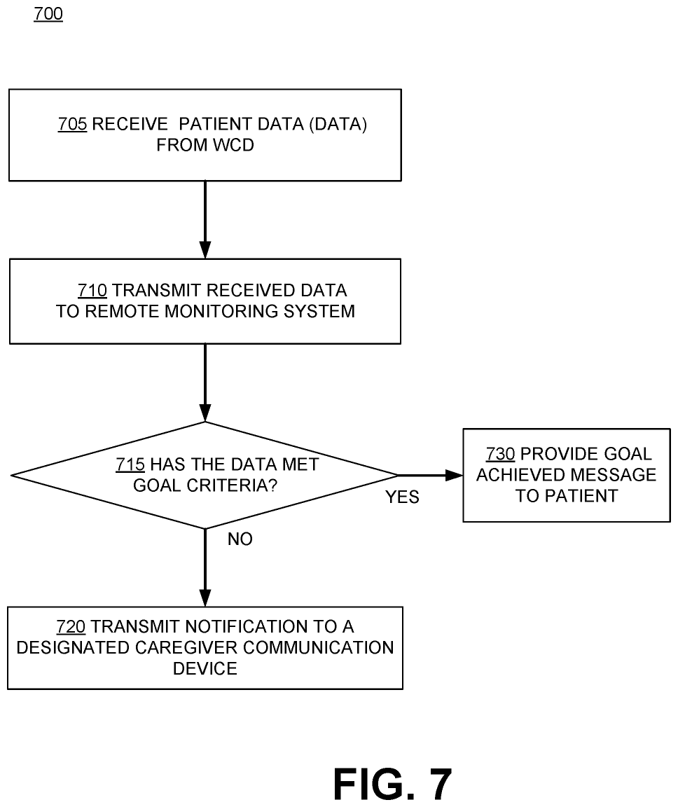

FIG. 7 is a flowchart illustrating a process 700, according to embodiments. In a step 705, patient data (“Data”) is received by a patient communication device. In some embodiments, the Data is received from a wearable medical device. In some embodiments, the patient communication device can be similar to, for example, the patient communication device 200 (FIG. 2) or the patient communication device 120 (FIG. 1). The wearable medical device can be a WCD similar to, for example, the WCD 100 (FIG. 1).

In a step 710, at least part of the received Data is transmitted to a remote monitoring system. In some embodiments, the Data is transmitted by the patient communication device, and the remote monitoring system can be similar to the remote monitoring system 150 (FIG. 1).

In a step 715, the transmitted Data is analyzed to determine that the patient has met the one or more goal criteria. In some embodiments, a memory (not shown) of the remote monitoring system is configured with the one or more goal criteria, and a processor (not shown) of the remote monitoring system is configured to analyze the transmitted data to determine that the patient has met the one or more goal criteria.

Upon determination that the patient has met the one or more goal criteria, the process 700 proceeds to a step 730 in which a message is sent to the patient communication device that a goal has been achieved. Such message can be displayed on the patient communication device.

However, if in step 715, it is determined that the one or more goal criteria have not been met, the process 700 proceeds to a step 720. In step 720, a notification is sent to a designated caregiver communication device. In some embodiments, the notification is sent by the remote monitoring system, and the notification informs a caregiver that the patient is not meeting the one or more goal criteria.

In the methods described above, each operation can be performed as an affirmative act or operation of doing, or causing to happen, what is written that can take place. Such doing or causing to happen can be by the whole system or device, or just one or more components of it. It will be recognized that the methods and the operations may be implemented in a number of ways, including using systems, devices and implementations described above. In addition, the order of operations is not constrained to what is shown, and different orders may be possible according to different embodiments. Examples of such alternate orderings may include overlapping, interleaved, interrupted, reordered, incremental, preparatory, supplemental, simultaneous, reverse, or other variant orderings, unless context dictates otherwise. Moreover, in certain embodiments, new operations may be added, or individual operations may be modified or deleted. The added operations can be, for example, from what is mentioned while primarily describing a different system, apparatus, device or method.

A person skilled in the art will be able to practice the present invention in view of this description, which is to be taken as a whole. Details have been included to provide a thorough understanding. In other instances, well-known aspects have not been described, in order to not obscure unnecessarily this description.

Some technologies or techniques described in this document may be known. Even then, however, it does not necessarily follow that it is known to apply such technologies or techniques as described in this document, or for the purposes described in this document.

This description includes one or more examples, but this fact does not limit how the invention may be practiced. Indeed, examples, instances, versions or embodiments of the invention may be practiced according to what is described, or yet differently, and also in conjunction with other present or future technologies. Other such embodiments include combinations and sub-combinations of features described herein, including for example, embodiments that are equivalent to the following: providing or applying a feature in a different order than in a described embodiment; extracting an individual feature from one embodiment and inserting such feature into another embodiment; removing one or more features from an embodiment; or both removing a feature from an embodiment and adding a feature extracted from another embodiment, while providing the features incorporated in such combinations and sub-combinations.

In general, the present disclosure reflects preferred embodiments of the invention. The attentive reader will note, however, that some aspects of the disclosed embodiments extend beyond the scope of the claims. To the respect that the disclosed embodiments indeed extend beyond the scope of the claims, the disclosed embodiments are to be considered supplementary background information and do not constitute definitions of the claimed invention.

In this document, the phrases “constructed to”, “adapted to” and/or “configured to” denote one or more actual states of construction, adaptation and/or configuration that is fundamentally tied to physical characteristics of the element or feature preceding these phrases and, as such, reach well beyond merely describing an intended use. Any such elements or features can be implemented in a number of ways, as will be apparent to a person skilled in the art after reviewing the present disclosure, beyond any examples shown in this document.

Incorporation by reference: References and citations to other documents, such as patents, patent applications, patent publications, journals, books, papers, web contents, have been made throughout this disclosure. All such documents are hereby incorporated herein by reference in their entirety for all purposes.

Parent patent applications: Any and all parent, grandparent, great-grandparent, etc. patent applications, whether mentioned in this document or in an Application Data Sheet (“ADS”) of this patent application, are hereby incorporated by reference herein as originally disclosed, including any priority claims made in those applications and any material incorporated by reference, to the extent such subject matter is not inconsistent herewith.

Reference numerals: In this description a single reference numeral may be used consistently to denote a single item, aspect, component, or process. Moreover, a further effort may have been made in the preparation of this description to use similar though not identical reference numerals to denote other versions or embodiments of an item, aspect, component or process that are identical or at least similar or related. Where made, such a further effort was not required, but was nevertheless made gratuitously so as to accelerate comprehension by the reader. Even where made in this document, such a further effort might not have been made completely consistently for all of the versions or embodiments that are made possible by this description. Accordingly, the description controls in defining an item, aspect, component or process, rather than its reference numeral. Any similarity in reference numerals may be used to infer a similarity in the text, but not to confuse aspects where the text or other context indicates otherwise.

The claims of this document define certain combinations and subcombinations of elements, features and acts or operations, which are regarded as novel and non-obvious. The claims also include elements, features and acts or operations that are equivalent to what is explicitly mentioned. Additional claims for other such combinations and subcombinations may be presented in this or a related document. These claims are intended to encompass within their scope all changes and modifications that are within the true spirit and scope of the subject matter described herein. The terms used herein, including in the claims, are generally intended as “open” terms. For example, the term “including” should be interpreted as “including but not limited to,” the term “having” should be interpreted as “having at least,” etc. If a specific number is ascribed to a claim recitation, this number is a minimum but not a maximum unless stated otherwise. For example, where a claim recites “a” component or “an” item, it means that the claim can have one or more of this component or this item.

In construing the claims of this document, the inventor(s) invoke 35 U.S.C. § 112(f) only when the words “means for” or “steps for” are expressly used in the claims. Accordingly, if these words are not used in a claim, then that claim is not intended to be construed by the inventor(s) in accordance with 35 U.S.C. § 112(f).

1-21. (canceled)

22. A method of providing care to a patient wearing a wearable cardioverter defibrillator (WCD), the method comprising:
processing, by a wearable cardioverter defibrillator processor, one or more signals sensed by the WCD into patient data;
transmitting by a communication module of the WCD the processed patient data to a patient communication device;
transmitting the processed patient data from the patient communication device to a remote monitoring system;
determining by the patient communication device that the processed patient data does not meet one or more of notification criteria; and
responsive to determining by the patient communication device that the processed patient data does not meet one or more of notification criteria, transmitting by the patient communication device a notification.

23. The method of claim 22, wherein the method further includes sending one or more messages by the remote monitoring system to the designated caregiver communication device, the one or more messages being different from the notification.

24. The method of claim 23, wherein the method further includes providing one or more instructions on how to provide care to the patient.

25. The method of claim 24, wherein providing the one or more instructions on how to provide care to the patient comprises instructions on how to check one or more of vital signs.

26. The method of claim 24, wherein providing the one or more instructions on how to provide care to the patient comprises instructions for troubleshooting a lack of WCD wear compliance.

27. The method of claim 24, wherein providing the one or more instructions on how to provide care to the patient comprises recommendations for troubleshooting a lack of step goal compliance.

28. The method of claim 24, wherein providing the one or more instructions on how to provide care to the patient comprises CPR instructions.

29. The method of claim 24, wherein providing the one or more instructions on how to provide care to the patient comprises a checklist.

30. The method of claim 24, wherein providing the one or more instructions on how to provide care to the patient comprises an instruction for the designated caregiver device to send a message to the remote monitoring system.

31. The method of claim 24, wherein providing the one or more instructions on how to provide care to the patient comprises one or more suggestions to encourage the patient reach a step count goal.

32. The method of claim 24, wherein providing the one or more instructions on how to provide care to the patient includes an augmented reality instruction.

33. The method of claim 22, wherein the one or more notification criteria comprise detection of an arrhythmia in the ECG data.

34. The method of claim 22, wherein the one or more notification criteria comprise the patient not taking a minimum number of steps per day.

35. The method of claim 22, wherein the one or more notification criteria comprise the patient not wearing the WCD at least 18 hours per day.

36. The method of claim 22, wherein the designated caregiver communication device comprises a designated caregiver notification application.

37. The method of claim 22, wherein the remote monitoring system is further configured to receive the notification and display the designated caregiver communication device location and the patient communication device location.

38. The method of claim 37, wherein the notification includes location information of the patient communication device.

39. The method of claim 37, wherein the remote monitoring system is configured to receive location information from the designated caregiver communication device.

40. The method of claim 39, wherein the remote monitoring system is configured to send a request for location information to the designated caregiver communication device in response to receiving the notification.
